SEQUENCE LISTING (2 sequences)

SEQ ID NO: 1 - protein, 17 aa, Artificial Sequence
  Description of Artificial Sequence Synthetic peptide designed for treating cardiac tamponade
ASGAPAPGPAGPLRPMF

SEQ ID NO: 2 - protein, 17 aa, Artificial Sequence
  Description of Artificial Sequence Synthetic peptide designed for treating cardiac tamponade
ASGAPAPGPAGPLRPMF

(19) United States
(12) Patent Application Publication
(10) Pub. No.: US 2016/0068571 A1
(43) Pub. Date: Mar. 10, 2016

(54) THERAPEUTIC AGENT FOR CARDIAC TAMPONADE

(71) Applicant: Research Institute for Protein Science Co., Nagoya-shi Aichi (JP)

(72) Inventors: Hidechika OKADA, Nagoya-shi (JP); Boros MIHALY, Szeged (HU); Erces DANIEL, Szeged (HU)

(21) Appl. No.: 14/762,378

(22) PCT Filed: Jan. 30, 2014
(86) PCT No.: PCT/JP2014/052015

§ 371 Date: Jul. 21, 2015

(30) Foreign Application Priority Data
Jan. 31, 2013 (JP) 2013-017409

(51) Int. Cl. C07K 7/08

(52) U.S. Cl. CPC C07K 7/08; A61K 38/00

(57) ABSTRACT

PROBLEM

To provide a therapeutic agent for cardiac tamponade to suppress occurrence of complications accompanying cardiac tamponade by preventing and improving deterioration of macro- and micro-circulation caused by cardiac tamponade and also by suppressing local and systemic inflammation accompanying such deterioration of circulation.

SOLUTION

A pharmaceutical which contains a peptide of the present invention or a pharmaceutically acceptable salt of such peptide as an active ingredient is useful as a therapeutic agent for cardiac tamponade (pericardial tamponade) caused by myocardial infarction, malignant tumor metastasis, collagen disease, or the like.

### FIELD OF THE INVENTION

The present invention generally relates to a therapeutic agent for cardiac tamponade and therapeutic method therefor. More particularly, the present invention relates to a therapeutic agent for cardiac tamponade and therapeutic method therefor comprising as an active ingredient a peptide having an activity of suppressing occurrence of complications accompanying cardiac tamponade by preventing and improving deterioration of macro- and micro-circulations caused by cardiac tamponade and also by suppressing local and systemic inflammation accompanying such deterioration of circulations.

### BACKGROUND ART

Cardiogenic shock is common among the patients developing cardiogenic or non-cardiogenic cardiac filling disorders like cardiac tamponade (for review see Non Patent Literature 1).

Pericardial tamponade caused by effusion of blood, pus, body fluid or air is a life-threatening state of medical emergency and therefore requires immediate active medical treatment (for review see Non Patent Literature 2). In such emergency case, the first choice is drainage of pericardial cavity by pericardial puncture or pericardiotomy.

Cardiogenic shock frequently causes low blood flow in macro-circulation and micro-circulation of internal organs, and subsequently thereto, activates local and systemic inflammation. It is widely accepted that acute insufficiency of myocardial pump causes complications with a high probability even after adequate treatment and activation of inflammation plays a decisive role under such circumstances.

However, the effectiveness of existing anti-inflammatory drugs for preventing or curing in vivo processes induced by low blood perfusion is extremely limited and therefore a new drug is required which, as supportive therapy, prevents occurrence of complications after or concurrently with relief of intrapericardial pressure caused by cardiac tamponade.

### PRIOR ART

### Non Patent Literature

NON PATENT LITERATURE 1: Topalian S, Ginsberg F, Parrillo J E: Cardiogenic shock. Crit Care Med 2008; 36:S66-S74

NON PATENT LITERATURE 2: Seferovic P M, Ristic A D, Imazio M, et al: Management strategies in pericardial emergencies. Herz 2006; 31:891-900

### SUMMARY OF THE INVENTION

### Technical Problem

The purpose of the present invention is to provide a therapeutic agent for cardiac tamponade to suppress occurrence of complications accompanying cardiac tamponade by preventing and improving deterioration of macro- and micro-circulation caused by cardiac tamponade and also by suppressing local and systemic inflammation accompanying such deterioration of circulation.

### SOLUTION TO PROBLEM

The inventors of the present invention found that a peptide consisting of the amino acid sequence of SEQ ID NO 2 of the Sequence Listing has an excellent curative effect for cardiac tamponade as a result of earnest investigation in order to solve the aforementioned problem and then have accomplished the present invention.

The present invention is:
(1) A therapeutic agent for cardiac tamponade comprising a peptide consisting of the amino acid sequence of SEQ ID NO. 2 of the Sequence Listing or its pharmaceutically allowable salt.
(2) The therapeutic agent for cardiac tamponade as described in (1) wherein dose per one time is in the range of 2 to 8 mg/kg.
(3) The therapeutic agent for cardiac tamponade as described in (1) or (2), wherein administration is made during the period of cardiac tamponade.
(4) A therapeutic method for cardiac tamponade comprising a step of administrating a peptide consisting of the amino acid sequence of SEQ ID NO. 2 of the Sequence Listing or its pharmaceutically allowable salt.
(5) The therapeutic method for cardiac tamponade as described in (4), wherein dose per one time is in the range of 2 to 8 mg/kg.
(6) The therapeutic method for cardiac tamponade as described in (4) or (5), wherein administration of a peptide consisting of the amino acid sequence of SEQ ID NO. 2 of the Sequence Listing or its pharmaceutically allowable salt is made during the period of cardiac tamponade.
(7) Use of a peptide consisting of the amino acid sequence of SEQ ID NO. 2 of the Sequence Listing or its pharmaceutically allowable salt in manufacture of a therapeutic agent for cardiac tamponade.

### ADVANTAGEOUS EFFECTS OF THE INVENTION

The present invention provides a peptide which is capable of treating cardiac tamponade by suppressing deterioration of systemic circulation and splanchnic microcirculation which may be a cause of complications causable by cardiac tamponade and local and systemic inflammation accompanying such deterioration of circulation.

### BRIEF DESCRIPTION OF THE DRAWINGS

FIG. 1A shows changes of mean arterial pressure in the sham-operated group (“Sham-operated” in FIG. 1A. The same shall apply hereafter), the non-treated group (“Tamponade” in FIG. 1A. The same shall apply hereafter) and the treated group (“Tamponade+AcPepA” in FIG. 1A. The same shall apply hereafter). FIG. 1B shows changes of cardiac output in the sham-operated group, the non-treated group and the treated group.

FIG. 2A shows changes of central venous pressure in the sham-operated group, the non-treated group and the treated group. FIG. 2B shows changes of the global end-diastolic volume index in the sham-operated group, the non-treated group and the treated group. FIG. 2C shows changes of heart rate in the sham-operated group, the non-treated group and the treated group.

FIG. 3A shows changes of superior mesenteric artery blood flow in the sham-operated group, the non-treated group and the treated group. FIG. 3B shows changes of average red blood cell verocity in the sham-operated group, the non-treated group and the treated group.

FIG. 4A shows changes of plasma histamine levels in the sham-operated group, the non-treated group and the treated group. FIG. 4B shows changes of plasma big-endothelin concentration in the sham-operated group, the non-treated group and the treated group. FIG. 4C shows changes of plasma HMGB-1 levels in the sham-operated group, the non-treated group and the treated group.

FIG. 5A shows changes of small intestinal myeloperoxidase activity in the sham-operated group, the non-treated group and the treated group. FIG. 5B shows changes of whole blood superoxide production in the sham-operated group, the non-treated group and the treated group.

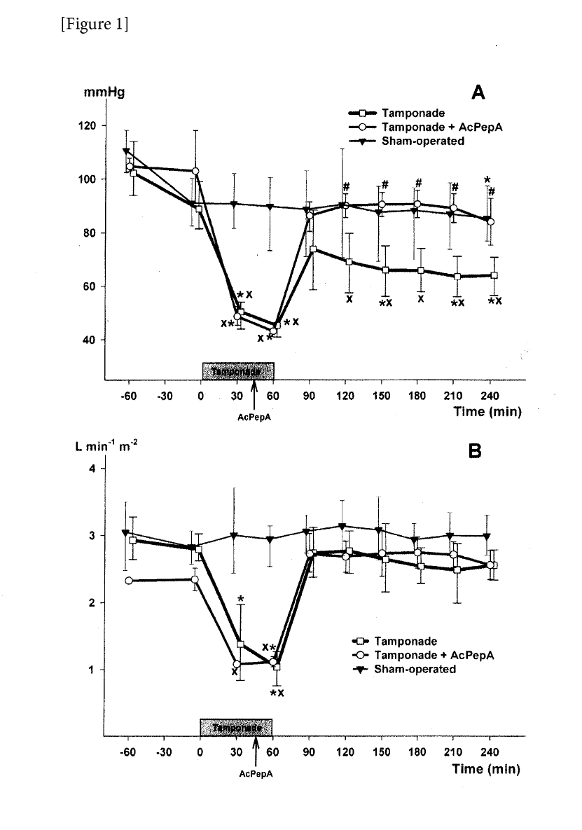

In all Figures (FIGS. 1A, 1B, 2A, 2B, 2C, 3A, 3B, 4A, 4B, 4C, 5A, 5B), the box (“Tamponade” is indicated therein) shows the duration of cardiac tamponade. The arrow shows the treatment with AcPepA.

The plots demonstrate the median values (transverse line in the rectangle) and the 25th and 75th percentile.

“*” indicates the significant level (p<0.05) within groups versus baseline values by Friedman Repeated Measures Analysis of Variance on Rank and Dunn's method following thereto. “x” indicates the significant level (p<0.05) between groups versus sham-operated group values by Kreskas-Wallis Analysis of Variance on Ranks and Dunn's method following thereto. “#” indicates the significant level (p<0.05) between treated groups versus non-treated group values by Kruskal-Wallis Analysis of Variance on Ranks and Dunn's method following thereto.

### DETAILED DESCRIPTION OF THE PREFERRED EMBODIMENTS

Preferred embodiments of the present invention are described in detail as below. However, each embodiment described below is an example of typical embodiments of the present invention. Accordingly, the scope of the present invention should not be restricted to the illustrated example beyond the scope of the appended claims.

The peptide of the present invention consists of the amino acid sequence of SEQ ID NO: 2 of the Sequence Listing wherein the amino group of N-terminal alanine is acetylated (herein also referred to as “AcPepA”). The peptide of the present invention is explained below. Such peptide has the structure wherein N-terminal of such peptide consisting of the amino acid sequence of SEQ ID NO: 1 of the Sequence Listing (herein also referred to as “PepA”) is acetylated, the sequence of which was designed by using the software named “MIMETIC program” (for review see Campbell W, Kleiman L, Barany L, Li Z, Khorchid A, Fujita E, et al. A novel genetic algorithm for designing mimetic peptides that interfere with the function of a target molecule. Microbiol Immunol. 2002; 46:211-5 and Baranyi L, Campbell W, Ohshima K, Fujimoto S, Boros M, Okada H. The antisense homology box: a new motif within proteins that encodes biologically active peptides. Nat Med. 1995; 1:894-901.), wherein genetic algorithm is employed for designing, by randomly changing amino acids, five thousands (5000) of peptide sequences which are expected to have high affinity to a target, and also points are assigned to each designed peptide sequence based on several physico-chemical parameters comprising optimization of complimentarity of the hydrophilic/hydrophobic index, optimization of the average structural similarity, minimization of side-chain interference and structural sequence, and besides by using original know-how, and also has higher stability than PepA.

The peptide of the present invention has the structure consisting of seventeen (17) amino acids (Ala Ser Gly Ala Pro Ala Pro Gly Pro Ala Gly Pro Leu Arg Pro Met Phe) wherein its N-terminal alanine is acetylated and each of constituent amino acids, Ala, Ser, Gly, Pro, Leu, Arg, Met and Phe, is natural type and indicate alanine, serin, glycin, proline, leucin, arginine, methionine and phenylalanine, respectively.

PepA may be prepared by solid-phase synthesis known per se (for review see Gerald F. singler, Anne K. Soutar, Louis C. Smith, Antonio M. Gotto, J R., James T. Sparrow. The solid phase synthesis of a protein activator for lecithin-cholesterol acyltransferase corresponding to human plasma apoC-I. Proc. Natl. Acad. Sci. USA, Vol. 73, No. 5, pp. 1422-1426, May 1976, Biochemistry), or by its equivalent method. On the other hand, the peptide of the present invention, AcPepA may be prepared from PepA by solid-phase synthesis known per se (for review see, for example, Atherton, E. and Sheppard, R. C. (1989) Solid phase peptide synthesis; a practical approach. IRL Press, Oxford. and Seikagakujikkenkoza (experimental lectures on biochemistry) tanpakushitunokagaku (science of protein) IV p 449, compiled by the Japanese Biochemical Society, Tokyokagakudojin (1977)), or by its equivalent method.

The pharmaceutically allowable salt of the peptide of the present invention means salt of inorganic acid such as hydrochloride, sulfate, hydrobromide, salt of organic acid such as acetate, citrate, methansulfonate, salt of inorganic base such as sodium salt, kalium salt and salt of organic base such as isopropylamine salt, 2-ethylaminoethanol salt.

In the present invention, “cardiac tamponade” (also referred to as “pericardial tamponade”) means a disease deriving from perforation or the like caused by uremia, metastasis of malignant tumor, cardiac infarction, collagen disease, trauma, aortic dissection, pericarditis or catheter surgery and also a disease which prevents expansion (beat) of the heart by blood or pericardial effusion staying in the pericardial cavity between the heart and the pericardium. The “period of cardiac tamponade” means the period of time when cardiac tamponade is developed.

In the present invention, “a therapeutic agent for cardiac tamponade” comprises, as an active ingredient, the aforementioned peptide (the peptide consisting of the amino acid sequence of SEQ ID NO. 2 of the Sequence Listing wherein the N-terminal alanine thereof is acetylated) or its pharmaceutically allowable salt, and may be used in the form of pharmaceutical preparation such as solid, semi-solid, liquid (for example, tablets, pellets, troches, capsules, suppository, creams, ointments, aerosols, powders, liquids, emulsions, suspensions, syrups and injections) or the like suitable for rectal, transnasal, pulmonary, transvaginal, external (local) or parenteral administration (including subcutaneous, implanted, intravenous and intramuscular administration). In the present invention, “a therapeutic agent for cardiac tamponade”, may be prepared by a routine procedure using various organic or inorganic carriers commonly used for a pharmaceutical purpose, for example, an excipient such as sucrose, starch, mannitol, sorbitol, lactose, glucose, cellulose, talc, calcium phosphate and calcium carbonate, a condensing agent such as cellulose, methyl cellulose, hydroxypropyl cellulose, polypropylpyrrolidone, gelatin, arabic gum, polyethylene glycol, sucrose and starch, a disintegrating agent such as starch, carboxymethyl cellulose, calcium carboxymethylcellulose, hydroxypropylstarch, sodium hydrogen carbonate, calcium phosphate and calcium citrate, a lubricating agent such as magnesium stearate, aerosol, talc and sodium lauryl sulfate, a flavoring agent such as citric acid, menthol, glycin and orange powder, a preservative such as sodium benzoate, sodium hydrogen sulfite, methylparaben and propylparaben, a stabilizing agent such as citric acid, sodium citrate and acetic acid, a suspending agent such as methyl cellulose, polyvinylpyrrolidone and aluminium stearate, a dispersing agent such as hydroxypropyl methyl cellulose, a diluting agent such as water and a base wax such as cacao butter, polyethylene glycol and white vaseline.

The dose of the peptide of the present invention varies depending upon route of administration, age of a patient, body weight, symptom, curative effect and the like and is administered, usually for an adult, once or several times a day at 0.5 mg-30 mg/kg per dose, preferably at 2 mg-8 mg/kg per dose.

The peptide of the present invention (AcPepA) may be used in the treatment of cardiac tamponade not only of human beings but also of animals other than human beings. Time of administration of the peptide of the present invention is particularly not restricted, but usually after development of cardiac tamponade, preferably during the period of cardiac tamponade.

In the therapeutic method for cardiac tamponade of the present invention, namely, in the therapeutic method for cardiac tamponade comprising a step of administrating a peptide consisting of the amino acid sequence of SEQ ID NO. 2 of the Sequence Listing or its pharmaceutically allowable salt, the peptide of the present invention (AcPepA) may be administered without particular limitation to mammals including human beings in the form of conventional pharmaceutical preparation aforementioned. Above all, intravenous administration, intramuscular administration or oral administration is preferable.

The peptide of the present invention may be used also as an intermediate in the manufacture of pharmaceuticals.

### EXAMPLES

The results of examination of the influences of AcPepA treatment on an early circulatory system and inflammatory changes by using clinically adequate acute porcine model of experimental cardiac tamponade are shown in examples. The present invention is explained more concretely by showing working examples. However, the present invention should not be restricted to the illustrated example.

### Example 1

Animals and Instrumentation

Inbred Vietnamese minipigs of both sexes (n=19, weighing 24±3 kg) were fasted for twelve (12) hours preoperatively, but received water ad libitum. Anesthesia was induced with a mixture of ketamine (20 mg/kg) and xylazine (2 mg/kg) im and maintained with a continuous infusion of propofol (50 μL/min/kg iv; 6 mg/kg/hr). After endotracheal intubation, the animals were mechanically ventilated with the tidal volume set at 9±2 mL/kg, and the respiratory rate was adjusted to maintain the end-tidal pressure of CO2 and PaCO2 in the range of 35 to 45 mmHg. Positive end-expiratory pressure was not applied during the period of cardiac tamponade.

The animals were placed in a supine position on an electric heating pad for maintenance of body temperature between 36 and 37° C., and received an infusion of Ringer's lactate at a rate of 10 mL/kg/h during the experiment. The right femoral artery and jugular vein were cannulated for the measurement of mean arterial pressure (“MAP”) and cardiac output (“CO”) by thermodilution (PICCO catheters, PULSION Medical Systems, Germany), and for fluid or drug administration, respectively. After a midline abdominal incision, the root of the superior mesenteric artery (“SMA”) was dissected free. An ultrasonic flow probe (Transonic Systems Inc., Ithaca, N.Y., U.S.A.) was placed around the exposed SMA to measure the mesenteric blood flow. In all protocols, the animals were monitored continuously, arterial blood gases were regularly checked (Cobas b121, Roche, Austria), and a period of 30 minutes was allowed for recovery from surgery.

The animals were randomly allocated to either of three experimental groups. Group 1 (n=6) served as the sham⁻operated group to which the same time-frame and sampling conditions as in the groups of 2 (n=7) and 3 (n=6) were applied, but without causing cardiac tamponade. Left lateral thoracotomy was performed in all groups. In the groups which developed cardiac tamponade, namely, in the group not treated with AcPepA (herein also referred to as “non-treated group”) and the group treated with AcPepA (herein also referred to as “treated group”), a cannula was fixed into the pericardial cavity. A pericardial tamponade was induced for 60 minutes by the intrapericardial administration of colloid solution, while the mean artery pressure was kept in the interval of 40 to 45 mmHg. After elapse of this period, the fluid was removed from the pericardial cavity and the animals were monitored for 180 minutes post-tamponade. Group 3 (the treated group) was treated with AcPepA (a single administration of 4 mg/kg in 5 mL saline iv into the jugular vein in a 5-minute infusion) after 45 minutes of cardiac tamponade. The beginning of tamponade is denoting 0 minute. Vehicle (saline) administration was applied in the groups of 1 and 2 by the same protocol.

Peripheral blood samples were taken, respectively, at baseline, after 75 minutes, after 150 minutes and at the end of the observation period (after 240 minutes) to detect levels of protein HMGB-1 (“High Mobility Group Box 1”), big-endothelin (“Big-ET”) and whole blood superoxide production. Small intestinal tissue biopsies were taken at the end of the experiment for myeloperoxidase (“MPO”) activity measurements.

Hemodynamic Measurements

Central venous pressure (“CVP”) and blood flow signals were continuously monitored and automatically registered with a computerized data-acquisition system (SPELL Haemosys; Experimetria Ltd., Budapest, Hungary). Mean arterial pressure, cardiac output and heart rate were measured with the PICCO Plus monitoring system (PULSION Medical Systems, Germany) and the global end-diastolic volume index (“GEDI”) was calculated with the PICCO system. Incidentally, central venous pressure was measured in each animal to exclude the influence of these factors, in parallel with the standard ventilation and fluid replacement protocols

C5A Antagonist Treatment

AcPepA (SEQ ID NO. 2 of the Sequence Listing) was synthetized and purified (>95% purity) by Research Institute for Protein Science Co. through acetylation of N-terminal alanine of PepA (ASGAPAPGPAGPLRPMF, SEQ ID NO. 1 of the Sequence Listing). The peptide was dissolved in saline and used in a concentration of 2 mg/mL (for review see Okada H, Imai M, Ono F, et al: Novel complementary peptides to target molecules. Anticancer Res 2011; 31:2511-2516).

### Intravital Videomicroscopy of the Microcirculation

An intravital orthogonal polarization spectral imaging technique (Cytoscan A/R, Cytometrics, Philadelphia, Pa., USA) was used for noninvasive visualization of the mucosal microcirculation of the small intestine. This technique utilizes reflected polarized light at the wavelength of the isobestic point of oxy- and deoxyhemoglobin (548 nm). As polarization is preserved in reflection, the only photons scattered from a depth of 200-300 μm contribute to image formation. A 10× objective lens was placed onto the serosal surface of the ascending colon, and microscopic images were recorded with an S-VHS video recorder 1 (Panasonic AG-TL 700, Matsushita Electric Ind. Co. Ltd, Osaka, Japan). Quantitative assessment of the microcirculatory parameters was performed off-line by frame-to-frame analysis of the videotaped images. Red blood cell velocity (“RBCV”, μm/s) changes in the postcapillary venules were measured in three separate visions by means of a computer-assisted image analysis system (IVM Pictron, Budapest, Hungary). All microcirculatory evaluations were performed by the same investigator.

Myeloperoxidase Activity

The activity of myeloperoxidase, a marker of activation of polymorphonuclear leukocyte, was measured by using ileal biopsy samples according to the method of Kuebler et al. (for review see Kuebler W M, Abels C, Schuerer L, et al: Measurement of neutrophil content in brain and lung tissue by a modified myeloperoxidase assay. Int J Microcirc Clin Exp 1996; 16:89-97). Briefly, a reaction mixture containing 50 mM K₃PO₄ buffer (pH 6.0), 2 mM 3,3′5,5′-tetramethylbenzidine (dissolved in DMSO) and 100 μL undiluted plasma sample was incubated for 5 minutes at 37° C. The reaction was started with 0.6 mM hydrogen peroxide (dissolved in 0.75 mL K₃PO₄ buffer) and was stopped after 5 minutes with 0.2 mL of H₂SO₄ (2 M), and the hydrogen peroxide-dependent oxidation of tetramethylbenzidine was detected spectrophotometrically at 450 nm (UV-1601 spectrophotometer, Shimadzu, Japan). Levels of myeloperoxidase were calculated via a calibration curve prepared with an myeloperoxidase standard (Sigma-Aldrich GmbH, Germany). To detect tissue myeloperoxidase activity, ileal samples were homogenized with Tris-HCl buffer (0.1 M, pH 7.4) containing 0.1 mM phenylmethanesulfonyl fluoride to block tissue proteases, and then centrifuged at 4° C. for 20 minutes at 24,000 g. Myeloperoxidase activities of such samples were measured as described above, and such data were referred to the protein content.

Whole Blood Superoxide Production

For measurement of the whole blood superoxide production, the chemiluminometric method of Zimmermann et al. was used (for review see Zimmermann T, Schuster R, Lauschke G, et al: chemiluminescence response of whole blood and separated blood-cells in case of experimentally induced pancreatitis and mdtq-da trasylol ascorbic-acid therapy. Anal Chimica Acta 1991; 255:373-381). During the measurements, 10 μL whole blood was added to 1 mL Hank's solution (PAA Cell Culture Company) and the mixture was kept at 37° C. until assay. The chemiluminometric response was measured with a Lumat LB9507 luminometer (Berthold, Germany) during a 30 minute period after addition of 100 μL of lucigenin.

Measurements of High-Mobility Group Box Protein-1, Big-Endothelin-1 and Histamine in Plasma

Four (4) ml of blood samples were drawn from the jugular vein into chilled polypropylene tubes containing EDTA (1 mg/mL), respectively, at baseline, after 75 minutes, after 150 minutes, and at the end of the observation period (after 240 minutes). Blood samples were centrifuged at 1200g for 10 minutes at 4° C. Next, plasma samples were collected and stored at −70° C. until assay.

Plasma concentrations of protein HMGB-1 were determined with a commercially available protein HMGB-1 ELISA kit (Shino-Test Corporation, Kanagawa, Japan). Plasma levels of big-endothelin, a 38 amino acid precursor protein of endothelin-1 (ET-1), were determined with a commercially available kit (Biochemica Hungaria Kft., Budapest, Hungary). Plasma histamine concentrations were determined by commercially available enzyme-linked immunoassay (Quantikine ultrasensitive EIA kit for histamine; Biomedica Hungaria Kft, Budapest, Hungary).

Statistical Analysis

Data analysis was performed with a statistical software package (SigmaStat for Windows (registered trademark); Jandel Scientific, Erkrath, Germany). Friedman repeated measures analysis of variance on ranks was applied within groups. Time-dependent differences from the baseline for each group were assessed by Dunn's method, and differences between groups were analyzed with Kruskal-Wallis one-way analysis of variance on ranks, followed by Dunn's method for pairwise multiple comparison. In each Figure, median values and 25th and 75th percentiles are given; p values<0.05 were considered significant.

Results

Macro-Hemodynamics

In the sham-operated group, there were no significant hemodynamic changes as compared with the baseline values, and mediator levels did not change significantly during observation period.

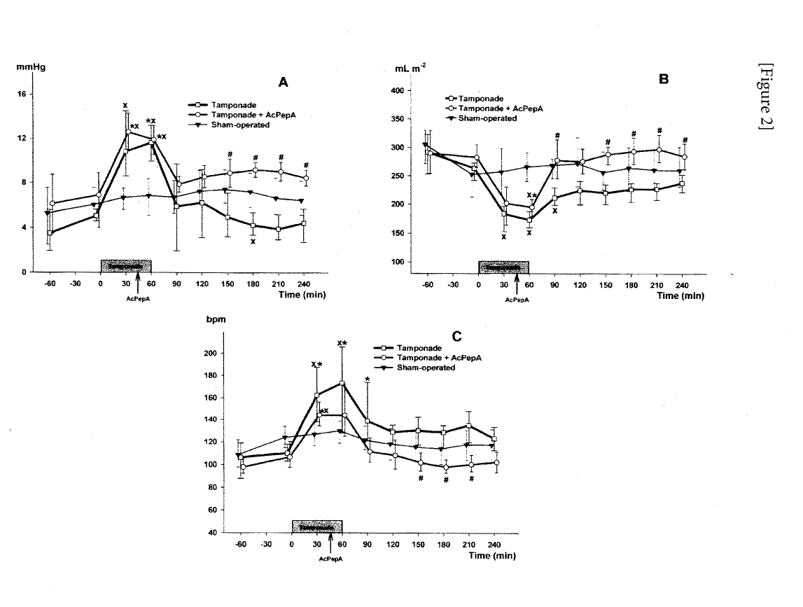

As shown in FIG. 1A, mean arterial pressure was kept in the interval of 40 to 45 mmHg during 60 minutes of cardiac tamponade by the infusion of colloid fluid into the pericardial cavity. As shown in FIG. 1B, this resulted in a significant 65% decline in cardiac output in both groups developing cardiac tamponade, namely, in the treated group and non-treated group and, as shown in FIG. 2C, a significant increase in heart rate, respectively, as compared with in the sham-operated group.

After relief of tamponade, mean arterial pressure was significantly lower in the non-treated group as compared with in the control group as shown in FIG. 1A, while cardiac output and heart rate returned to the baseline values despite such reduction in mean arterial pressure as shown, respectively, in FIGS. 1B and 2C. On the other hand, mean arterial pressure increased significantly in the treated group and showed significantly high values as compared with in the non-treated group and also showed the same values as in the sham-operated group. However, as shown, respectively, in FIGS. 1B and 2C, cardiac output was not affected and heart rate showed significant decrease in the post-tamponade period as compared with in the non-treated group. These facts show that AcPepA has the effect of preventing occurrence of complications accompanying cardiac tamponade by significantly suppressing the decrease of mean arterial pressure after relief of cardiac tamponade and also maintaining such mean arterial pressure in the same state as that where cardiac tamponade is not developed and by realizing good myocardial blood circulation and oxygen supply through maintenance of lower heart rate.

As shown in FIG. 2A, the significant increase of central venous pressure was observed during the period of cardiac tamponade in the treated group and non-treated group as compared with in the sham-operated group, which apparently shows decline in the venous return. As shown in FIG. 2B, a significant decrease of the global end-diastolic volume index occurs at the same time in the treated group and non-treated group as compared with in the the sham-operated group.

As shown in FIG. 2A, central venous pressure showed a decreasing trend after relief of cardiac tamponade in the treated group and non-treated group. As shown in FIG. 2B, the global end-diastolic volume index showed significant low values in the non-treated group as compared with in the treated group and did not reach the base-line values. These changes observed in the non-treated group demonstrate the long-lasting impairment of the venous return flow following the cardiac tamponade. On the other hand, both central venous pressure and the global end-diastolic volume index in the treated group showed significant high values as compared with in the non-treated group and also showed the same values as in the sham-operated group. Such significant high values of the global end-diastolic volume index indicate that the return blood flow increased significantly as compared with in the non-treatment group. These facts show that AcPepA has the effect of remarkably preventing occurrence of the long-lasting impairment of the venous return flow that may cause complications after development of cardiac tamponade and also restoring the venous return flow to the state before development of cardiac tamponade.

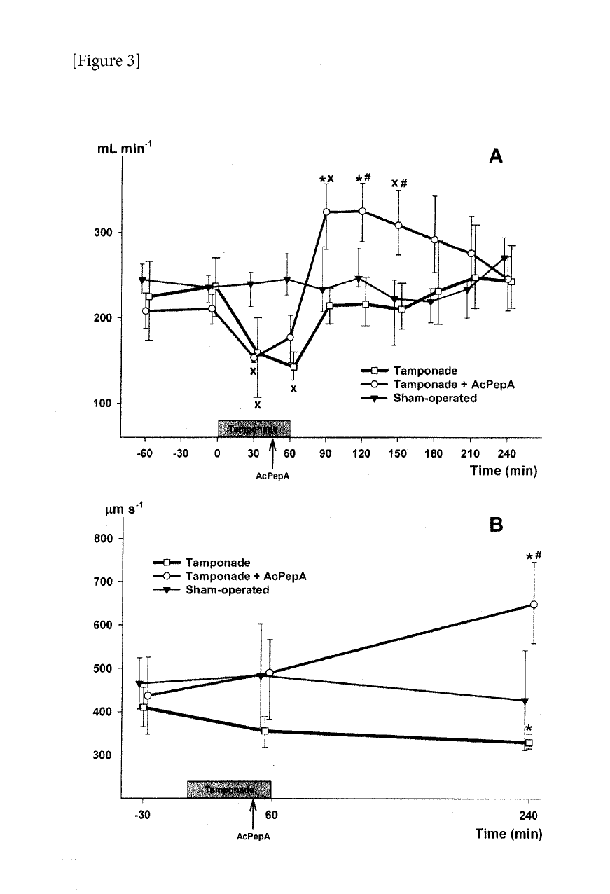

As shown in FIG. 3A, a significant decrease in the superior mesenteric artery blood flow was observed during the period of cardiac tamponade in the treated group and non-treated group as compared with in the sham-operated group, which indicates that redistribution deteriorated the mesenteric circulation. On the other hand, after relief of cardiac tamponade, the superior mesenteric artery blood flow returned to the control values in the treated group and non-treated group and, as the result, deterioration of the mesenteric circulation caused by the aforementioned redistribution was eliminated. Further, it was observed that the superior mesenteric artery blood flow in the treated group increased significantly after relief of tamponade as compared with in the sham-operated group. These facts show that AcPepA has the effect of preventing occurrence of complications by improving the splanchnic circulation through correction of delay in blood flow recovery of the superior mesenteric artery that may cause complications after development of cardiac tamponade.

Microcirculation

A heterogeneous oscillating microcirculation was found in the small intestinal mucosa in all groups, and therefore the weighted average of red blood cell verocity (herein referred to as “average red blood cell verocity”) was calculated. The average red blood cell verocity was calculated on the basis of the duration of the fast-flow and slow-flow periods and the red blood cell verocity during the respective phases (for review see Szabo A, Suki B, Csonka E, et al: Flow motion in the intestinal villi during hemorrhagic shock: A new method to characterize the microcirculatory changes. Shock 2004; 21: 320-328).

As shown in FIG. 3B, the average red blood cell verocity showed no change in the sham-operated group, but in the non-treated group a decreasing trend from the baseline values was observed at the end of the cardiac tamponade and then a significant decrease was shown at the end of the experiment in comparison with the baseline level. At the end of the post-tamponade phase, in the treated group the average red blood cell verocity was increased significantly from the baseline level and also as compared with in the non-treated group.

Biochemical Parameters

Peripheral blood samples were taken for measurement, respectively, at baseline, after 75 minutes, after 150 minutes and at the end of the observation period (after 240 minutes).

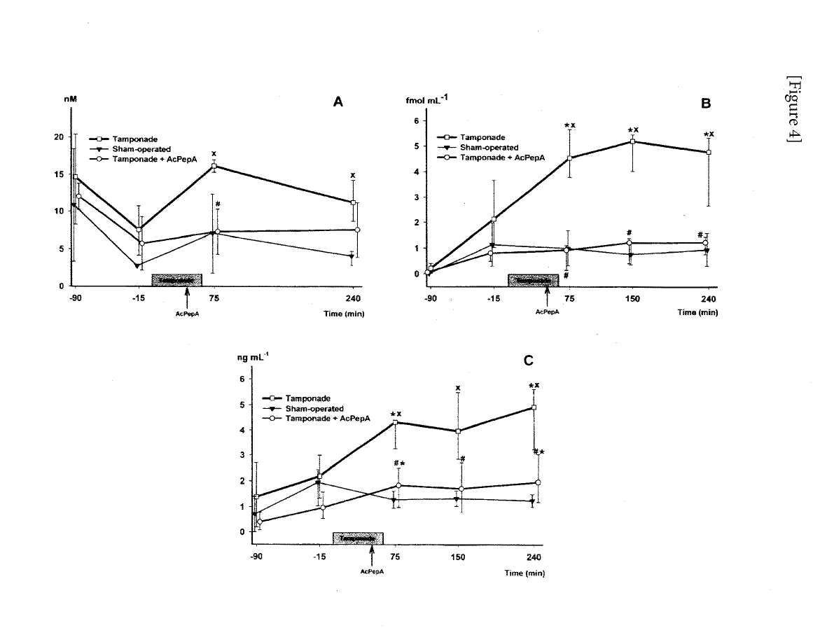

As shown in FIG. 4A, as a result of cardiac tamponade, histamine levels were increased significantly in the non-treated group at 15 minutes of the post-tamponade phase as compared with in the sham-operated group. On the other hand, histamine levels in blood plasma were decreased significantly in the treated group as compared with in the non-treated group and showed the same values as in the sham-operated group.

Big-endothelin is a stable precursor of endothelin-1 with a longer half-life that is released from different types of cells. Plasma big-endothelin levels increased significantly 4- to 5-fold after cardiac tamponade in the non-treated group as compared with in the sham-operated group as shown in FIG. 4B. On the other hand, plasma big-endothelin levels in the treated group showed low values as compared with in the non-treated group and also showed the same values as in the sham-operated group.

Plasma levels of protein HMGB-1, a very effective signal for leukocyte activation which causes an escalation of the inflammatory process, was elevated significantly in the non-treated group after the compression of the heart from the baseline level and as compared with in the sham-operated group as shown in FIG. 4C. On the other hand, Plasma levels of protein HMGB-1 in the treated group showed significant low values as compared with in the non-treated group and also showed the same values as in the sham-operated group.

These facts show that AcPepA has the effect of preventing occurrence of complications of cardiac tamponade by suppressing release of vasoactive and proinflammatory mediators including histamine, protein HMGB-1 and big-endothelin accompanying acute hemodynamic changes occurring in the cardiac tamponade and by contributing to the increase of venous return.

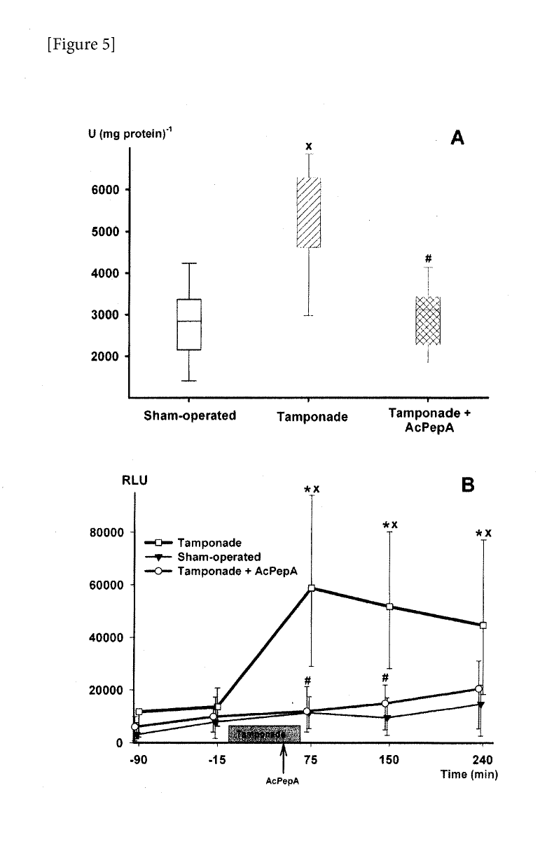

The rate of polymorphonuclear leukocyte accumulation was determined through measurement of myeloperoxidase activity in the small intestine tissue samples taken at the end of the experiment. As shown in FIG. 5A, Myeloperoxidase activity was significantly higher in the non-treated group as compared with in the sham-operated group, which indicates the increased accumulation of polymorphonuclear leukocytes. On the other hand, polymorphonuclear leukocyte activity showed significant low values in the treated group as compared with in the non-treated group and also showed the same values as in the sham-operated group. Such significant low values indidate the decreased accumulation of polymorphonuclear leukocytes.

In the blood at early stage of the post-tamponade phase, as shown in FIG. 5B, it was observed that superoxide production was significantly increased in the non-treated group as compared with in the sham-operated group, and that values of superoxide production in the treated group were low as compared with in the non-treated group and also showed the same values as in the sham-operated group.

These facts show that AcPepA enables prevention of occurrence of complications of cardiac tamponade through improvement of microcirculation resulting from maintenance of endothelial function by suppressing deterioration of systemic circulation, subsequent occurrence of similar disorders in the splanchnic microcirculation and increase of myeloperoxidase activity entrained thereafter, and then by decreasing causes of tissue damage.

### INDUSTRIAL APPLICABILITY

The peptide relating to the present invention (AcPepA) is available to the therapy of cardiac tamponade and contributable to the development of such industrial field, because AcPepA enables prevention of occurrence of complications caused by cardiac tamponade.

1. A therapeutic agent for cardiac tamponade comprising a peptide consisting of the amino acid sequence of SEQ ID NO. 2 of the Sequence Listing or its pharmaceutically allowable salt.

2. The therapeutic agent for cardiac tamponade as claimed in claim 1, wherein dose per one time is in the range of 2 to 8 mg/kg.

3. The therapeutic agent for cardiac tamponade as claimed in claim 1, wherein administration is made during the period of cardiac tamponade.

4. A therapeutic method for cardiac tamponade comprising a step of administrating a peptide consisting of the amino acid sequence of SEQ ID NO. 2 of the Sequence Listing or its pharmaceutically allowable salt.

5. The therapeutic method for cardiac tamponade as claimed in claim 4, wherein dose per one time is in the range of 2 to 8 mg/kg.

6. The therapeutic method for cardiac tamponade as claimed in claim 4, wherein administration of a peptide consisting of the amino acid sequence of SEQ ID NO. 2 of the Sequence Listing or its pharmaceutically allowable salt is made during the period of cardiac tamponade.

7. Use of a peptide consisting of the amino acid sequence of SEQ ID NO. 2 of the Sequence Listing or its pharmaceutically allowable salt in manufacture of a therapeutic agent for cardiac tamponade.
